(19) United States
(12) Patent Application Publication
(10) Pub. No.: US 2009/0131532 A1
(43) Pub. Date: May 21, 2009

(54) Method of preemptive treatment of inflammation, algesia and poor healing using topical capsaicin

(75) Inventor: James L. Flowers, Las Vegas, NV (US)

Correspondence Address: RYAN KROMHOLZ & MANION, S.C., POST OFFICE BOX 26618, MILWAUKEE, WI, 53226, US

(73) Assignee: MFM Medical Enterprises

(21) Appl. No.: 12/290,948

(22) Filed: Nov. 5, 2008

Related U.S. Application Data
(60) Provisional application No. 61/002,076, filed on Nov. 6, 2007.

(51) Int. Cl. A61K 31/164; A61P 29/00

(52) U.S. Cl. 514/627

(57) ABSTRACT

A method of treating the conditions which involve inflammation, algesia and ineffective healing by preemptively applying capsaicin topically prior to or at the onset of the process thereby diminishing harmful effects.

### RELATED APPLICATIONS

This application claims the benefit of co-pending U.S. provisional patent application Ser. No. 61/002,076, filed on 6 Nov. 2007.

### FIELD OF INVENTION

The present invention relates to a method of preventing or diminishing the untoward effects of pathological processes by applying a medication to skin before or at the onset of the process.

### BACKGROUND OF THE INVENTION

The traditional manner of treating most conditions is to treat them after the process has occurred. There are well known efforts at prevention of pathological conditions. This is best exemplified by the use of vaccines which result in immunity to the offending infectious agents. There are also attempts at prevention of complications of conditions by controlling the pathological process. An example is the control of hypertension to prevent the complications of hypertension such as strokes. Similar efforts occur in diabetes management. These last two methods have been extraordinarily successful. The success not only relates to the decrease in the unwanted conditions or complications, but also is associated with minimal and acceptable side effects. Other conditions have not been as successful in preventive efforts. Rheumatoid arthritis is a very disabling condition. The medications which have been used to slow the disease down and to try to prevent complications have been only partially successful and have had serious side effects. These medications include gold, penicillamine, methotrexate, Enbrel and prednisone among others. The destruction of rheumatoid arthritis is closely tied to the rampant inflammation which results. Conditions known to result in pain and or scarring such as burns or surgery also require treatment after the event has occurred. Poor healing and pain may also be related to the inflammation which occurs. Pain is also tied to the nerves serving the affected area. Chronic pain is especially tied to changes in the nerves serving the affected area. The initial injury, such as a burn or surgical incision, can cause changes in the sensory nerves which may be transmitted centrally to the central nervous system (CNS), which can result in gene and chemical changes which cause the prolonged pain syndrome. The treatment of chronic pain is very difficult and often unsuccessful. Attempts have been made to try to pre-empt the pain post-operatively, for example, by premedicating the patient with narcotics and or non-steroidal anti-inflammatory agents (NSAID's). To date, the success has been mixed but promising. All of these methods require the systemic administration of the medications. The availability of a medication which can actually decrease or prevent the extent of inflammation and pain resulting from a process, which would be inexpensive, which would be easy to apply and could be targeted to the site of the process without systemic absorption and have minimal side effects would be a tremendous advance. These benefits do result from this invention.

Capsaicin (trans-8-methyl-N-vanillyl-6-nonenamide), the pungent principle of red peppers, has been used cutaneously for the treatment of pain. Synthetic capsaicin (nonyl vanillylamide) acts in the same manner as capsaicin, and capsaicin (more properly capsaicinoids) is meant to include this substance for brevity. Capsaicin is believed to act on a subset of primary afferent nerves mostly of the C fiber type (polymodal, thin, unmyelinated), although some A delta (thin, myelinated) are also capsaicin sensitive. Capsaicin is believed to bind to a receptor at the nerve ending and cause a release of a variety of neuropeptides (e.g., substance P, CGRP and others) and results in afferent nerve conduction initially. That receptor has been recently identified and designated as vanillyl receptor 1. There are two main phases of action, first an excitation and then a desensitization of the nerve to stimulation. The excitation results in the ‘hot’ of hot pepper or the burning tingling sensation which can occur when capsaicin is applied to the skin. The desensitization results from the depletion of neuropeptides and interference with afferent conduction in a non-tetrodotoxin dependent manner. It has been demonstrated that topically (skin) applied capsaicin can deplete neuropeptides at the peripheral (cutaneous) and central (dorsal horn of spinal cord) endings of the peripheral cutaneous nerve. C fibers have also been connected to some inflammatory processes. To date patents and articles have only described the use of capsaicin to treat cutaneous conditions (e.g., psoriasis or post-herpetic neuralgia), neuropathic conditions affecting the skin or subcutaneous area (e.g., post-mastectomy neuroma or diabetic neuropathy) or direct subcutaneous conditions (e.g., osteoarthritis or rheumatoid arthritis). All of these conditions have been treated for the purpose of pain relief, and the medication has only been used after the condition has been present or the pain, inflammation or complications have occurred. I am aware of no use of capsaicin to treat any of these except as described.

The present invention is related to the inventor's earlier related works, U.S. patent application Ser. No. 07/870,510 filed Apr. 17, 1992, now U.S. Pat. No. 5,178,879, and U.S. patent application Ser. No. 08/000,752 filed Jan. 5, 1993, now abandoned, and U.S. patent application Ser. No. 08/213,654, filed Mar. 16, 1994 now U.S. Pat. No. 5,431,914 which are incorporated herein by reference.

### SUMMARY OF INVENTION

It is the object of this invention to disclose a method which comprises externally applying capsaicin to the skin or mucous membrane to prevent the onset and or progression of conditions or processes which may ordinarily result from the condition. Specifically, the prevention of excessive inflammation, the prevention of excessive pain and the allowance of more rapid and complete healing. It must be understood that this is distinctly different and novel from the current approach to the above conditions. Typically, it is only after the pain has occurred, after the inflammation has occurred or after healing is impaired or a scar has resulted does the intervention occur. It is the essence of this invention that the unwanted effects of a condition are controlled or prevented by application of the capsaicin before such unwanted effects occur. For example, can capsaicin, applied to the future site of a burn, prevent or decrease the inflammation, prevent or decrease the pain and act to increase the healing rate or completeness? of course, medication can be given afterwards to decrease the pain, such as narcotics or NSAID's. Medication can be given afterwards to decrease some of the inflammation, such as NSAID's. There are still no recognized medications which will increase the rate or completeness of healing.

Through a combination of general medical and pharmacological knowledge, through extensive work with capsaicin, through an understanding of the newer discoveries of how the nervous system functions in relation to pain and inflammation and healing, and through fortuitous clinical uses of capsaicin, this invention was born. It is not obvious that a medication which is used to treat a condition once it is in place will be useful or even proper before the onset of the process. There are situations in medicine in which medication is given before certain procedures or in certain situations to decrease the chance of an event occurring. This is best understood with the use of antibiotics. Whereas, antibiotics are most often given after an infection is present, such as pneumonia, they are given preemptively in certain situations where the risk of infection is high. For example, in individuals known to have a damaged heart valve, and who will be undergoing an invasive tooth/gum procedure, prophylactic antibiotics are routinely given. This method depends on adequate levels of antibiotic in the system at the time of the procedure to kill any microorganisms which may be transiently present. It is actually a direct treatment of the offending organism which is the usual use of the antibiotic. But for most medications, even for those which could be argued to have a preventive effect, they are not administered unless the condition is present. No one takes high blood pressure pills before they have been diagnosed with high blood pressure. There is no reason to believe that by taking a high blood pressure pill, there will be a prevention of the onset of high blood pressure. The same is true of diabetes. No one takes insulin to prevent diabetes even though it is very effective once the diagnosis is made. Also, no-one takes an ibuprofen before there is evidence of inflammation or pain. No one takes morphine before the onset of the pain. There are no known creams which are applied to a normal area of skin to prevent a scar from forming there. So, although, there are the rare uses of a medicine before the onset of a condition, this is uncommon. There has been no reason to expect the pre-emptive application of capsaicin would be effective in the situations herein.

The idea began when a patient suffered a burn against the hot eye of a stove. Usually, this is very painful and results in good size blister. Capsaicin gel was immediately applied, within the first minute. The result was a minimum of pain, as the patient had done this many times before, and there was virtually no blistering. A burn is a good model to test this hypothesis because burns have active inflammation, shown by the blister and pain among other signs, have significant pain, undergo a healing process and may leave a scar. The inflammation of burns, although different in some respects, is similar to the inflammation of rheumatoid and other arthritidites and to inflammation caused by surgical incisions and other lacerations. Inflammation is a generic process to injury throughout the body. Multiple processes initiate it, e.g. a burn or arthritis or an incision, but the process of it and its outcome are often very similar. Uncontrolled inflammation will lead to pathology. This occurs in arthritis, in some painful conditions and can contribute to poor and delayed healing, or scar formation. If the process of inflammation can be controlled, all of the above, the pain and the healing, may be controlled. Also, pain may be controlled if the nerves signaling pain, the C fibers, are controlled.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention.

### Definition of Terms

I adopt the definition of terms for the structures of the capsaicinoids as described in the document, “Method of Treating an Internal Condition by External Application of Capsaicin Without the Need For Systemic Absorption” U.S. Pat. No. 5,431,914, which is incorporated herein by reference.

CAPSAICIN, NATURAL—Capsaicin is derived from the fruits of the Solanaceae family and the Capsicum genus. The crude isolate of the fruit is called capsicum oleoresin and contains over 100 chemicals. A further extraction process results in ‘natural capsaicin’. This isolate contains up to 5 related molecules which are capsaicin, dihydrocapsaicin, nordihydrocapsaicin, homocapsaicin and homodihydrocapsaicin 1. Most of this ‘natural capsaicin’ is made of capsaicin and dihydrocapsaicin. The ‘natural capsaicin’ was used in our trials. When capsaicin (more properly capsaicinoids) is used herein, it is meant to include these five molecules and synthetic capsaicin as described below. The compounds are:

CAPSAICIN (N-[(4-hydroxy-3-methoxyphenyl)methyl]-8-methyl-(E)-6-nonenamide) or (trans-8-methyl-N-vanillyl-6-nonenamide)

### CAS REGISTRY NUMBER—404-86-4

### MOLECULAR FORMULA—C₁₈H₂₇NO₃

### MOLECULAR WEIGHT—305.4 amu

DIHYDROCAPSAICIN (N-[(4-hydroxy-3-methoxyphenyl)methyl]8-methylnonanamide)

### CAS REGISTRY NUMBER—19408-84-5

### MOLECULAR FORMULA—C₁₈H₂₉NO₃

### MOLECULAR WEIGHT—307.4 amu

CAPSAIClNOIDS—The capsaicinoids are compounds with action like capsaicin. These include the natural products (under Capsaicin, Natural) and the synthetic (under Capsaicin, Synthetic).
CAPSAICIN, SYNTHETIC—Synthetic capsaicin is present in the natural plants but in small amounts. It is sold as a capsaicin substitute. It also affects the CSPA's just like capsaicin. It has been used in our trials and has been effective. For convenience, the term capsaicin (actually capsaicinoids) used herein is meant to encompass both the natural capsaicin as discussed above and the synthetic capsaicin. The details for ‘synthetic capsaicin’ are:
NONIVAMIDE (N-[(4-hydroxy-methoxyphenyl)methyl]nonanamide)
OTHER COMMON NAMES—Pelargonic acid vanillylamide Nonylic acid

### CAS REGISTRY NUMBER—2444-46-4

### MOLECULAR FORMULA—C₁₇H₂₇NO₃

### MOLECULAR WEIGHT—293.4 amu

CAPSAICIN when used by itself includes capsaicin, natural; capsaicinoids; and capsaicin, synthetic.

### Experimental Data in Support of Invention

An experiment was conducted to test the hypothesis that capsaicin might prevent inflammation, pain and poor healing in a pre-emptive manner. The author was the experimental human volunteer.

### Experimental Design:

The study was double blinded with one subject. Three substances, water, gel vehicle and capsaicin 0.025% (w/w) gel were applied to three designated areas on the back of the subject. These applications were done three times a day for one week. The assistants making these applications, as well as the subject himself, were blinded to the compositions of the gel preparations. After the one week, a different assistant applied a series of burns with the tip of a curling iron to the locations of the applications. The temperature of the curling iron was 56° C. Prior testing had demonstrated this would result in a significant second degree burn (blisters) to the subject. Two burns within a few minutes were administered to the sites of the prior applications.

### Outcome Measurements

Over a one-week period after administering the burns, observations were recorded, by an assistant blinded to the substances which were applied. Then observations were continued over the next 24 months. The parameters measured were the extent of blister formation, the amount of pain experienced and the extent and degree of healing.

### Results of the Experiment

The pain was much less for the capsaicin 0.025% than for the water. The blister formed was the least for the capsaicin 0.025% than for the water or the vehicle. The healing, evaluated over months, was significantly better for the capsaicin 0.025% than for the water or the vehicle. These findings were evident over the first week and persisted over the 33 months. The final evaluation of the burns took place 33 months after the initial burn. At that time, the following was found: the worst site was the site of an over the counter popular rub on pain reliever. This site was judged worse due to its appearance which still looked and, especially, felt like a blister, was irregular and was bumpy. The capsaicin sites, whether applied prior to the burn or just after the burn, were much better than the water or the over counter rub on preparation. The capsaicin site, 0.075% (w/w) applied after the burn, was slightly better than the 0.025% site applied before the burn demonstrating the effectiveness of applying the capsaicin immediately after the injury. This site was also homogenous in tone, was flat and was nearly the same color as the skin.

### Discussion of the Experiment

Although, this was limited to one subject, the results clearly showed that capsaicin applied before the onset of the pathological process, the burn, did decrease the full extent of the complications of the process, i.e. the inflammation, pain and poor healing. Based on the clinical observation above, it is felt that the capsaicin will also be effective if applied within minutes after a process has occurred. There was another arm in the study in which capsaicin 0.075% was applied immediately after the burn was placed (the second best site after 33 months). This also resulted in some decreased pain, less of a blister and enhanced healing as compared to the water preparation.

In the preferred practice of the method of the present invention a gel containing a safe and effective amount of capsaicin is topically applied to the skin or mucous membrane of the area of the process or pathological condition. This may be applied before the process has its onset, immediately after the onset, or, in the case of chronic conditions, after the process has its onset but before unwanted complications.

### Description of Clinical Uses of the Invention

### Burn Treatment:

It is usually not possible to predict the timing or location of a burn. But, a first aide kit may contain capsaicin which is applied within minutes after the onset of the burn. For burns which are first and second degree and some third degree, it is expected to result in less pain, blistering and better healing. The capsaicin is best applied before the onset of blistering or pain. Other agents known to decrease the effects of burn may be added such as aloe vera, benzocaine, or other topical analgesics, or topical antibiotics without affecting the essence of the invention.

Inflammatory Arthritidies:

These conditions may be diagnosed before the onset of the crippling complications occurs with bone and soft tissue destruction. So, although the diagnosis has been made, the devastating part of the process has yet to occur. The application of capsaicin, not to treat existing pain or inflammation, but to preemptively prevent the onset or extent of inflammation and pain would be the course of treatment. Note carefully that this is not what is being currently done for arthritis. There is neither medical literature nor FDA recognition of the use of capsaicin in this manner—this is entirely novel and is the essence of this invention.

Surgical Healing and Post-Op Pain:

Capsaicin is applied to the intended site of the surgical incision before the incision is made. The capsaicin will decrease the amount of inflammation, decrease post-op pain and will enhance healing.

Other Conditions:

It is claimed that this method of capsaicin will be effective in any condition in which inflammation is a key factor and the afferent C fibers are involved in terms of the inflammation and/or pain and/or sensory mediation. The identification and use of capsaicin is only limited by the novelty of this invention. Many other conditions will be identified which are treatable by this method.

The optimum length of time in which the capsaicin is applied prior to the onset of the process has not been determined. The time may vary from immediately after the event to months before the event. In the case of chronic conditions the capsaicin may be used continuously. Typically, it may be applied four times a day initially, and after desensitization has occurred it may be applied as little as once a day.

Any topical preparation containing a concentration of capsaicin from about 0.01% to 0.10% (w/w) can be used. Higher concentrations are mainly limited by the burning side effects and it becomes a rubifacient/vesicant and counterirritant. The method described does not use nor depend on the counterirritant effects of capsaicin. In fact, the counterirritant effects would obviate its use in the manner described. Preparations containing 0.025% to 0.075% (w/w) capsaicin are preferred. The preparation may be a gel, cream, ointment, suspension, solution, spray, lotion, roll-on, gel stick, patch, paste, plaster or other topical type of vehicle or preparation (e.g., as part of a cold or hot pack, as ultrasound or iontophoresis or phonophoresis).

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention.

I/We claim:

1. A method of treating burns to preemptively decrease inflammation, pain and enhance healing, comprising the steps of:
topically applying a solution comprising capsaicin from 0.01% to 0.10% (w/w).

2. The method of claim 1 in which the capsaicin solution is applied from 1 to 4 times a day beginning at a period ranging from 3 months before to immediately before the burn.

3. The method of claim 1 in which the capsaicin solution is applied within 60 minutes of the formation of the burn.

4. The method of claim 1 in which the capsaicin solution is chosen from one of the following: gel, cream, ointment, suspension, solution, spray, lotion, roll-on, gel stick, patch, paste, plaster or other topical type of vehicle or preparation, as part of a cold or hot pack, as ultrasound or iontophoresis or phonophoresis.

5. A method of treating inflammatory arthritidies, such as rheumatoid arthritis, to preemptively decrease inflammation, and its consequences, and pain, comprising the steps of:
topically applying a solution comprising capsaicin from 0.01% to 0.10% (w/w).

6. The method of claim 5 in which the capsaicin solution is applied from 1 to 4 times a day.

7. The method of claim 5 in which the capsaicin vehicle is chosen from one of the following: gel, cream, ointment, suspension, solution, spray, lotion, roll-on, gel stick, patch, paste, plaster or other topical type of vehicle or preparation, as part of a cold or hot pack, as ultrasound or iontophoresis or phonophoresis.

8. A method of treating the post-op problems of surgical incisions including inflammation, pain and poor healing (not related to infectious complications) comprising the steps of:
topically applying a solution comprising capsaicin from 0.01% to 0.10% (w/w).

9. The method of claim 8 in which the capsaicin solution is applied from 1 to 4 times a day beginning at a period ranging from 3 months before to immediately before the incision.

10. The method of claim 8 in which the capsaicin solution is applied within 60 minutes after the closure of the incision.

11. The method of claim 8 in which the capsaicin vehicle is chosen from one of the following: gel, cream, ointment, suspension, solution, spray, lotion, roll-on, gel stick, patch, paste, plaster or other topical type of vehicle or preparation (e.g., as part of a cold or hot pack, as ultrasound or iontophoresis or phonophoresis).
